(19) United States
(12) Patent Application Publication
(10) Pub. No.: US 2001/0048890 A1
(43) Pub. Date: Dec. 6, 2001

(54) REMOVAL METHOD OF MALODOROUS SUBSTANCE AND DEODORIZATION DEVICE THEREOF

(76) Inventor: NAOAKI SATA, TOKYO (JP)

Correspondence Address: SUGHRUE MION ZINN MACPEAK AND SEAS, 2100 PENNSYLVANIA AVENUE NW, WASHINGTON, DC, 20037

(21) Appl. No.: 09/187,926

(22) Filed: Nov. 6, 1998

(30) Foreign Application Priority Data
Nov. 6, 1997 (JP) 303846/1997

(51) Int. Cl. A61L 9/00

(52) U.S. Cl. 422/5; 422/122; 422/180; 422/171; 423/213.7; 423/230; 423/244.06; 423/244.04; 423/244.1

(57) ABSTRACT

A removal method of malodorous substance and deodorization device thereof enables un-treated gas containing soluble or insoluble malodorous substance contained complexly to be removed by only one treatment process as far as the degree of sensing nothing by organoleptic test. The un-treated gas containing soluble or insoluble malodorous substance contained complexly is flushed in compliance with necessity to remove insoluble malodorous substance, before converting insoluble malodorous substance into soluble substance by virtue of conversion catalyst to flush to be removed.

### BACKGROUND OF THE INVENTION

The present invention relates to a removal method of water insoluble malodorous substance. More to particularly, this invention relates to a removal method of malodorous substance and deodorization device thereof in which the removal method and the device enable removal of malodorous substance which can not be treated individually to be executed on the ground that there is combined a means for dissolving water insoluble malodorous substance in water with a means for flushing to be removed the dissolved malodorous substance.

Description of the Related Art

By way of the removal method for the stench, there are the washing method, the absorption method, the catalytic oxidation method, and the photocatalytic decomposition method and so forth. To remove the stench was implemented from a considerable time ago using above respective methods.

The washing method is to remove the stench component such that it causes the stench gasses to be washed using appropriate solvent. Water is used most frequently by way of solvent. In the method using water simply, the stench substance which is very readily soluble in water such as ammonia can be removed, however, it is difficult to remove the stench substance which is not readily soluble in water, for instance, dimethylsulfide and so forth. For this reason, Japanese Patent Publication No. SHO 61-22605 discloses a technology in which water added hypochlorite is used for washing treatment of the above dimethylsulfide. However, such additives are of the high price, so that it is of economically disadvantage. The additives are disagreeable in excessive state or in insufficient state. For this reason, it is always necessary to watch concentration of the un-treated substrate, thus it becomes necessary to prepare a large-scale monitor. As described above, to use the additives is to interfere with simplicity of the device and of the economical advantage which are of the advantages of the washing method. When there is only one kind of stench substance to be removed, and it can find the appropriate additives corresponding thereto, it is capable of being answered thereto. When the stench substances are of more than two kinds, the effective additives in respective stench substances behave with each other, or challenge in reaction with the stench substance, thus there might occur the case where the effective removal becomes impossible.

The absorption method is the method for removing the malodorous substance by means of appropriate absorbent or absorption material. Zeolite and so forth are very frequently used as the absorption material. For instance, Japanese Patent Application Laid-Open No. HEI 8-257105 discloses removal method of mercaptan and so forth and deodorant material regenerative technology by means of hydrophobic deodorant material consisting of hydrophobic zeolite supported by metal oxides. However, when there is implemented the treatment of methanethiol (mercaptan) or regeneration thereof, power of oxidization of the deodorant material deteriorates, because sulfur is performed occlusion by way of form of sulfur monoxide or sulfur dioxide. Another problem of the absorption method is that the limit exists in possible absorption quantity of the untreated substance. The un-treated substance and absorption material which is saturated with the intermediate product for oxidization should be discarded or regenerated. The handling of both cases is troublesome, and to discard them is to bring further economical disadvantage. Furthermore, in the case of regeneration, the method which can regenerate it completely is not many. When the regeneration is incomplete, it is discarded ultimately, thus coming to be exchanged for new absorption material.

Catalytic oxidation method is the method for converting malodorous substance which is subjected to air oxidation using catalyst into non-malodorous substance. For instance, the Japanese Patent Application Laid-Open HEI 7-284670 discloses deodorization removal technology using catalyst whose effective component is active manganese dioxide and so forth. According to the preceding technology, there is known that methanethiol and so forth can be removed at most only degree of 90%. In general, relationship between concentration of stench substance and intensity of stench (hereinafter referring to stench intensity) sensed by human nose due to the organoleptic test becomes relationship of logarithm (Law of Weber Fechner). For instance, in many cases, even though the concentration of the stench substance comes to be 1/10, the stench intensity comes to be only degree of ½. Consequently, since the above technology can remove stench substance incompletely, it is of no practical use.

Photocatalytic decomposition method is to decompose to be treated the stench substance using photocatalyst. For instance, Japanese Patent Application Laid-Open No. HEI 9-75434 discloses deodorization technology of mercaptans and so forth using photocatalyst consisting of porous absorbent supported by titanium dioxide, ultraviolet sensitizer, transparent ceramics, and transparent binder and so forth. In the literature, there is supposed that 500 ppm of methanethiol is capable of being treated completely after time period of 2 hours using the photocatalyst. However, it is too slow to require 2 hours as far as complete removal, thus it is not practical. Furthermore, ammonia, acetaldehyde, acetic acid, are incapable of being removed completely after 3 to 4 hours treatment. Thus photocatalytic reaction whose reaction is generally slow is not practically in continuous treatment of the stench substance.

It is possible to remove with respect to ammonia, hydrocarbons, lower aldehyde and so forth by using singly appropriate one of the above described prior art. However, there is no description in the embodiment of the above respective technology in terms of removal of higher aldehydes with strong stench. Further, in terms of sulfur-containing stench substances such as dimethylsulfide or methanethiol or the like, it is incapable of being removed thoroughly. Even though it can be removed, it takes a lot of time. There occurs another stench component by way of intermediate product of decomposition. There occurs lowering of deodorizing function. Moreover, the technology which shows remarkable effect to the specified stench substance is capable of narrow application to the another stench substance. Thus such technology is practically disadvantage because stench gas contains many stench components.

It might be capable of combining two kinds or three kinds of conventional technology which are logically available. The absorption method and catalytic oxidation method are combined most frequently, (For instance, Japanese Patent Application Laid-Open No. HEI 7-100327, Japanese Patent Application Laid-Open No. HEI 9-187624), also there is proposed combination between the absorption method and the photocatalytic decomposition method (For instance, Japanese Patent Application Laid-Open No. HEI 8-259344 and so forth). However, these are to use catalysts for simplifying regenerative process in the only absorption method being essential qualities of the technology, thus it is incapable of being obtained the effect more than combination of respective component technology.

The above described higher aldehydes and sulfur-containing compounds are water insoluble, therefore it is difficult to remove completely by the flushing method. It is difficult to remove and to decompose completely by the oxidation catalyst, photocatalyst, or absorption material. In the case of simple combination thereof, for instance, when combining catalytic oxidation method with the flushing method with water as solvent, it is capable of being removed the substance capable of decomposing by catalyst and soluble malodorous substance, however, higher aldehyde or sulfur-containing compounds which are insoluble and can not be decomposed by deodorization catalyst can not be removed.

### SUMMARY OF THE INVENTION

In view of the foregoing, it is an object of the present invention for achieving the above mentioned problem to provide a removal method of malodorous substance and deodorization device thereof in which soluble or insoluble stench substance contained complexly is capable of being removed by only one treatment process as far as the degree of sensing nothing by organoleptic test.

In one arrangement to be described below by way of example in illustration of the invention, a removal method of malodorous substance, which comprises the steps of converting insoluble malodorous substance within untreated substance containing both of soluble malodorous substance and insoluble malodorous substance into soluble substance by virtue of catalyst (hereinafter referring to conversion catalyst) enabling the malodorous substance being insoluble and having non-decomposition property in treatment of deodorizing catalyst to be converted into soluble substance, and flushing to be removed the soluble substance. Namely, the present invention is the invention for removal method of malodorous substance characterized by the fact that there is converted insoluble malodorous substance into soluble substance by virtue of catalyst, then flushing to be removed the malodorous substances.

By way of the conversion catalyst in the present invention, the catalyst of Co-Al₂O₃ is capable of being used in relation to higher aldehydes which is difficult to be removed by the catalyst used for flushing treatment or conventional deodorization treatment. The Mo—Co catalysts are capable of being used in relation to thiols or dialkyl sulfides. The Pt catalysts are capable of being used in relation to higher ester group. The Pt catalysts or Pd catalysts are capable of being used in relation to aromatic hydrocarbons. The Pt catalysts or SiO₂—Al₂O₃ catalysts are capable of being used in relation to aliphatic hydrocarbons. These conversion catalysts have action for oxidizing malodorous substance under existence of air. These conversion catalysts further advance oxidative decomposition according to treatment condition, so that the catalysts have action enabling the malodorous substance to be converted into soluble substance of low-molecule.

In many cases, stench gasses are mixed gasses consisting of various malodorous components. The stench gasses consisting of only malodorous substances being insoluble and having non-decomposition property in treatment of deodorizing catalyst belong to, if anything, exception. In many cases, there are contained insoluble and soluble malodorous substances, and the malodorous substances which are capable of being decomposed easily by conventional deodorizing catalysts. The present invention is capable of being applied to the stench gasses.

It is capable of enhancing deodorizing efficiency on the ground that the conversion catalyst and the deodorizing catalyst are combined in answer to necessity to be used to the stench gasses containing malodorous substance capable of being decomposed by the deodorizing catalyst. In regard to combination between deodorizing catalyst and conversion catalyst, there are two cases either the deodorization catalyst and the conversion catalyst are mixed or respective individual catalyst layers are arranged. A concrete configuration of the combination would be selected so as to remove the malodorous substance effectively.

A target of the present invention is the insoluble malodorous substance which can not be decomposed by the deodorization catalyst, and with the exception of another limitation. Consequently, effective combination between the deodorization catalyst and the conversion catalyst conflicts on the malodorous substance to be target. There are two combinations in that one series arrangement causes untreated substance to contact with the deodorization catalyst previously, and to contact with the conversion catalyst afterward, and the other series arrangement causes untreated substance to contact with the conversion catalyst previously, and to contact with the deodorization catalyst afterward. Furthermore, when there exists catalyst which combines a deodorization function and a conversion function, it is suitable to use it instead of the above described mixed catalysts.

Moreover, when untreated substance contains insoluble substance and soluble substance, it is suitable to remove soluble malodorous substances beforehand, while arranging the flushing process in front of the catalyst deodorization process in order to decrease load to the catalyst. It is capable of further improving absorption efficiency by adding various kinds of auxiliary absorbent in this flushing process.

It is capable of using well-known tower-vessel group type apparatus singly or with combined.

The above and further objects and novel features of the invention will more fully understood from the following detailed description when the same is read in connection with the accompanying drawing. It should be expressly understood, however, that the drawing is for purpose of illustration only and is not intended as a definition of the limits of the invention.

### BRIEF DESCRIPTION OF THE DRAWING

FIG. 1 is a system view showing a embodiment of a method according to the present invention.

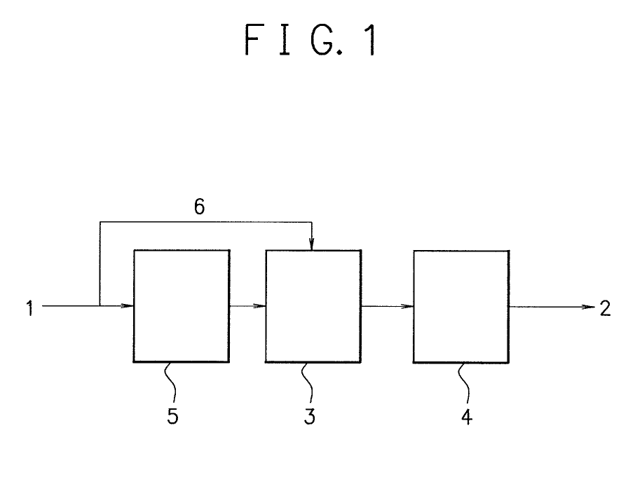

### DETAILED DESCRIPTION OF THE PREFERRED EMBODIMENT

A preferred embodiment of the invention will be described. In the present invention, any one of conversion catalyst which causes un-treated malodorous substance to be soluble is available. The present invention is not limited by any kind of the conversion catalysts. The most suitable catalyst conflicts on a target in accordance with corresponding relationship to kinds of un-treated malodorous substance. For instance, the effective conversion catalyst against the above described higher aldehyde is Co—Al₂O₃ catalysts. The effective conversion catalyst against sulfur-containing compounds is Mn—Co catalyst.

Further, the flushing method used in the present invention is not restricted particularly, it is capable of being selected the well-known effective method appropriately in accordance with the form and concentration of the malodorous substance after contact treatment of catalyst. It is also capable of being selected the well known effective method appropriately in cases where flushing method is used as former process, thus the flushing method is not restricted particularly. Water is the most suitable by way of solvent. According to circumstances, it is suitable to add solvent to water.

Hereinafter, the detail of the present invention will be described referring to the embodiments. In the case of without notice, “%” represents weight %, gas concentration is represented by capacity criterion ppm.

[Embodiment 1]

Gamma-alumina (BET ratio surface area: approximately 150 m²/g) is subjected to impregnation into aqueous solution of cobalt chloride (CoCl₂) at 40° C. during time period of 24 hours, before performing reduction under hydrogen current of air at 220° C., during 4 hours, thus obtaining catalyst (hereinafter the conversion catalyst is referred to Co/Al₂O₃) in which gamma-alumina is supported by 0.5%-cobalt. There is arranged in series a filled layer of Co/Al₂O₃ catalyst in upper stream, and flushing device of water in downstream. There was treated imitated malodorous air containing 100 ppm of butanal in the ventilation condition of SV/h⁻¹=10,000. Concentration of butanal after treatment became 0 ppm. The treatment was implemented over a time period of 200 hours. There was no deterioration of removal capability of butanal.

### COMPARISON EXAMPLE 1

The imitated malodorous air which is used in the above embodiment 1 was treated by only catalyst of Co/Al₂O₃ in the condition of SV/h⁻¹=10,000. The concentration of butanal became 0 ppm. However concentration of butanoic acid became 69 ppm, result in no removal of the stench.

### COMPARISON EXAMPLE 2

The imitated malodorous air which is used in the above embodiment 1 was treated by only flushing device. The concentration of butanal decreases to 24 ppm by way of numeral value, however butanal could not be removed by way of the stench.

[Embodiment 2]

The embodiment 2 was implemented using the same catalyst and device as that of the embodiment 1. There was treated imitated malodorous air containing 100 ppm of pentanal in the ventilation condition of SV/h⁻¹=10,000. Concentration of pentanal after treatment became 0 ppm. The treatment was implemented over the time period of 200 hours. There was no deterioration of removal capability of pentanal.

### COMPARISON EXAMPLE 3

The imitated malodorous air which is used in the above embodiment 2 was treated by only catalyst of Co/Al₂O₃ in the condition of SV/h⁻¹=10,000. The concentration of pentanal became 3 ppm. However the concentration of pentanoic acid became 49 ppm, result in no removal of the stench.

### COMPARISON EXAMPLE 4

The imitated malodorous air which is used in the above embodiment 2 was treated by only flushing device. The concentration of pentanal decreases to 12 ppm by way of numeral value, however pentanal could not be removed by way of the stench.

[Embodiment 3]

There is arranged in series a filled layer of catalyst (hereinafter conversion catalyst is referred to Mn—Co catalyst) obtained in such a way that manganese dioxide (MnO₂) and cobalt oxide (CoO) are mixed with each other in the condition of weight ratio of 95:5 in upper stream, and flushing device of water in downstream. There was treated imitated malodorous air containing 100 ppm of diethyl sulfide in the ventilation condition of SV/h⁻¹=10,000. Concentration of diethyl sulfide after treatment became 0 ppm. The treatment was implemented over a time period of 200 hours. There was no deterioration of removal capability of diethylsulfide.

### COMPARISON EXAMPLE 5

The imitated malodorous air which is used in the above embodiment 3 was treated by only catalyst of Mn—Co in the condition of SV/h⁻¹=10,000. The concentration of diethylsulfide became 0 ppm. However the concentration of sulfur dioxide became 46 ppm, result in no removal of the stench.

### COMPARISON EXAMPLE 6

The imitated malodorous air which is used in the above embodiment 3 was treated by only flushing device. The concentration of diethylsulfide decreases to 26 ppm by way of numeral value, however diethylsulfide could not be removed by way of the stench.

[Embodiment 4]

The embodiment 4 was implemented using the same catalyst and device as that of the embodiment 3 namely filled layer of Mn—Co catalyst and flushing device are arranged in series. There was treated imitated malodorous air containing 100 ppm of dimethylsulfide in the ventilation condition of SV/h⁻¹=10,000. Concentration of dimethylsulfide after treatment became 0 ppm. The treatment was implemented over a time period of 200 hours. There was no deterioration of removal capability of dimethylsulfide.

### COMPARISON EXAMPLE 7

The imitated malodorous air which is used in the above embodiment 4 was treated by only catalyst of Mn—Co in the condition of SV/h⁻¹=10,000. The concentration of dimethylsulfide became 0 ppm. However the concentration of sulfur dioxide became 56 ppm, result in no removal of the stench.

### COMPARISON EXAMPLE 8

The imitated malodorous air which is used in the above embodiment 4 was treated by only flushing device. The concentration of dimethylsulfide decreases to 19 ppm by way of numeral value, however dimethylsulfide could not be removed by way of the stench.

[Embodiment 5]

Filled layer of Mn—Co catalyst and flushing device are arranged in series. There was treated imitated malodorous air containing 100 ppm of methanethiol in the ventilation condition of SV/h⁻¹=10,000. Concentration of methanethiol after treatment became 0 ppm. The treatment was implemented over the time period of 200 hours. There was no deterioration of removal capability of methanethiol.

### COMPARISON EXAMPLE 9

The imitated malodorous air which is used in the above embodiment 5 was treated by only catalyst of Mn—Co in the condition of SV/h⁻¹=10,000. The concentration of methanethiol became 0 ppm. However the concentration of sulfur dioxide became 39 ppm, result in no removal of the stench.

### COMPARISON EXAMPLE 10

The imitated malodorous air which is used in the above embodiment 5 was treated by only flushing device. The concentration of methanethiol decreases to 27 ppm by way of numeral value, however methanethiol could not be removed by way of the stench.

[Embodiment 6]

There are arranged in series a gas flushing tower, next a catalyst tower filled with catalyst (hereinafter referring to Pt-catalyst) in which gamma-alumina is supported by Pt of 0.3% due to impregnation method, and next a gas flushing tower. There was treated imitated malodorous air containing each 30 ppm of propanoic acid, ethanoic acid, butyl ethanoate, toluene, and cyclohexane in the ventilation condition of SV/h⁻¹=10,000. Concentration of all malodorous substances after treatment became 0 ppm. The treatment was implemented over a time period of 200 hours. There was no deterioration of removal capability of the malodorous substances.

### COMPARISON EXAMPLE 11

The imitated malodorous air which is used in the above embodiment 6 was treated by only the Pt-catalyst tower in the condition of SV/h⁻¹=10,000. The concentration of propane acid and ethane acid became 18 ppm and 14 ppm respectively. Result in no removal of the stench.

### COMPARISON EXAMPLE 12

The imitated malodorous air which is used in the above embodiment 6 was treated by only flushing device. The concentration of propanoic acid, ethanoic acid, butyl ethanoate, toluene, and cyclohexane became 6 ppm, 2 ppm, 16 ppm, 18 ppm, 11 ppm respectively, thus the stench is not removed.

As described above, according to the present invention, malodorous substance such as higher aldehydes or sulfur-containing compounds which are incapable of being treated easily by means of deodorizing catalyst of the conventional technology, and which are of insoluble and are of hardly decomposition are capable of being treated effectively to be deodorized.

While preferred embodiments of the invention have been described using specific terms, such description is for illustrative purpose only, and it is to be understood that changes and variations may be made without departing from the spirit or scope of the following claims.

What is claimed is:

1. A removal method of malodorous substance comprising the steps of:
converting insoluble malodorous substance within un-treated substance containing soluble malodorous substance and insoluble malodorous substance into soluble substance by virtue of catalyst; and
flushing to be removed said soluble substance.

2. A removal method of malodorous substance as claimed in claim 1, wherein there is taken one together a catalyst for converting insoluble malodorous substance into soluble substance and a catalyst for deodorizing a stench by way of said catalyst.

3. A removal method of malodorous substance as claimed in claim 1, wherein it causes insoluble malodorous substance to be separated from said untreated substance, then converting insoluble malodorous substance into soluble substance, before flushing to be removed said soluble substance.

4. A removal method of malodorous substance as claimed in claim 2, wherein it causes insoluble malodorous substance to be absorbed from said un-treated substance, before converting insoluble malodorous substance separated from absorbent into soluble substance.

5. A removal method of malodorous substance as claimed in any one of claims 1 to 4, wherein said catalyst which converts insoluble malodorous substance into soluble substance is oxidative decomposition catalyst.

6. A removal method of malodorous substance as claimed in claim 5, wherein it causes insoluble malodorous substance to be converted into soluble substance by said catalyst under existence of air.

7. A removal method of malodorous substance as claimed in any one of claims 1, 2, 3, or 6, wherein there is used catalyst in which gamma-alumina is supported by cobalt by way of said catalyst.

8. A removal method of malodorous substance as claimed in any one of claims 1, 2, 3, or 6, wherein there is used catalyst in which gamma-alumina is supported by platinum by way of said catalyst.

9. A removal method of malodorous substance as claimed in any one of claims 1, 2, 3, or 6, wherein there is used catalyst in which gamma-alumina is supported by palladium by way of said catalyst.

10. A removal method of malodorous substance as claimed in any one of claims 1, 2, 3, or 6, wherein there is used catalyst of silica-alumina system by way of said catalyst.

11. A removal method of malodorous substance as claimed in any one of claims 1, 2, 3, or 6, wherein there is used catalyst whose effective component is manganese dioxide (MnO₂) and cobalt oxide (CoO) by way of said catalyst.

12. A deodorization device for deodorizing a gas containing malodorous substance continuously comprising:
a catalyst layer for converting insoluble malodorous substance into soluble substance; and
a flushing removal section for removing soluble malodorous substance provided at downstream thereof.

13. A deodorizing device as claimed in claim 12, wherein there is provided a flushing removal section for removing insoluble malodorous substance added at upper stream of said catalyst layer for converting said insoluble malodorous substance into soluble substance.
